(19) United States
(12) Patent Application Publication
(10) Pub. No.: US 2026/0295001 A1
(43) Pub. Date: Oct. 1, 2026

(54) COMPOSITIONS AND METHODS FOR TREATMENT OF SYSTEMIC LUPUS ERYTHEMATOSUS

(71) Applicant: Dan GOLDSTAUB, Even Yehuda (IL)

(72) Inventor: Dan GOLDSTAUB, Even Yehuda (IL)

(21) Appl. No.: 19/498,649

(22) PCT Filed: Jul. 3, 2024
(86) PCT No.: PCT/IL2024/050648

§ 371 Date: Dec. 31, 2025

Related U.S. Application Data
(60) Provisional application No. 63/524,834, filed on Jul. 3, 2023.

(51) Int. Cl. A61K 38/10; A61K 9/00; A61K 47/69; A61K 48/00; A61P 37/06; C12N 15/85

(52) U.S. Cl. CPC A61K 38/10; A61K 9/0019; A61K 47/6901; A61K 48/0041; A61K 48/005; A61K 48/0075; A61P 37/06; C12N 15/85

(57) ABSTRACT

The present invention relates to a microbe or microbe like particle comprising a peptide having an SLE therapeutic efficacy, an analog or a fragment thereof, wherein the peptide is displayed on the surface of the microbe or microbe like particle.

### SEQUENCE LISTING STATEMENT

The instant application contains a Sequence Listing which has been submitted electronically in XML format and is hereby incorporated by reference in its entirety. Said XML copy, created on Jun. 26, 2023, is named 16301-PC Sequence Listings.xml and is 27 Kilobytes in size.

### TECHNICAL FIELD OF THE INVENTION

The present invention is in the field of pharmaceutical compositions and methods of treatment of a disease. In particular, the invention relates to drug delivery systems for treating systemic lupus erythematosus (SLE) and to peptides effective in the treatment of SLE.

### BACKGROUND OF THE INVENTION

Systemic lupus erythematosus (SLE) is an autoimmune disease caused by the ‘self-attack’ of the immune system against body antigens, resulting in inflammation and tissue damage. It can manifest in a chronic manner or be of a form that has recurrent relapses. SLE is characterized by the presence of an array of autoantibodies, including antibodies to DNA, antibodies to nuclear antigens, and antibodies to ribonucleoproteins. It has the potential of affecting multiple organ systems including the skin, muscles, bones, lungs, kidneys, cardiovascular and central nervous systems. Renal complications, infections, myocardial infarction and central nervous system involvement are the major causes of morbidity and even death in SLE patients.

To date there is no definitive treatment or cure for SLE which is devoid of detrimental side effects associated with immunosuppression. In recent years some peptide-based therapies for SLE have been developed and clinically tested.

An artificial peptide (a.k.a. “consensus” peptide [pCONS]) was developed using an algorithm that defines the T cell stimulatory amino acid sequences from the VH region of multiple BWF1 IgG antibodies to DNA. The peptide illustrated a delay in the appearance of autoantibodies and prolonged survival in mice. Bevra H. Hahn et al. Arthritis & Rheumatism, Vol. 44, No. 2, February 2001, pp 432-441.

International Patent Application Publication No. WO 1996/030057 discloses synthetic peptides based on a complementarity-determining region (CDR) of the heavy or light chain of a pathogenic anti-DNA monoclonal antibody that induces a systemic lupus erythematosus (SLE)-like disease in mice, and pharmaceutical compositions comprising said peptides for the treatment of SLE in humans.

International Patent Application Publication No. WO 2002/067848 discloses peptides based on the complementarity-determining region of the human monoclonal anti-DNA 16/6Id antibody capable of immunomodulating SLE associated responses. In particular, peptides based on the CDR1 and CDR3 of the human 16/6Id were found to inhibit the proliferative response of peripheral blood lymphocytes (PBL) of SLE patients to the human anti-DNA 16/6ld mAB, and to ameliorate disease manifestations of mice afflicted with spontaneous or experimental SLE.

International Patent Application Publication Nos. WO 2004/064788 and WO 2004/064787 disclose aqueous pharmaceutical compositions of peptides derived from the complementarity-determining region of the human monoclonal anti-DNA 16/6Id.

Peptide based therapies are known to be efficacious but also suffer from some unfavorable characteristics mainly regarding their pharmacokinetic behavior, including plasma stability, membrane permeability and circulation half-life.

In recent years an immense advancement has been made in the field of drug delivery, inter alia, in microbe-based drug delivery systems for treating various diseases. Viral nanoparticles (VNPs) encompass a diverse array of naturally occurring nanomaterials that can encapsulate active ingredients or be genetically or chemically conjugated to such active ingredients. Microbe-based drug delivery systems are manufactured through scalable fermentation or molecular farming and are biocompatible and biodegradable. These properties have led to a wide range of applications, including cancer therapies, immunotherapies, vaccines, antimicrobial therapies, cardiovascular therapies, gene therapies, as well as imaging and theragnostic (Young Hun Chung et al. Adv Drug Deliv Rev. 2020; 156:214-235).

There is a need for new and effective delivery systems of peptides that will provide enhanced retention and efficacy in treating SLE.

### SUMMARY OF THE INVENTION

In one aspect, the present invention provides a microbe-based drug delivery system comprising a microbe or microbe like particle; and a peptide having a systemic lupus erythematosus (SLE) therapeutic efficacy, wherein said peptide is displayed on the surface of said microbe or microbe like particle.

In one or more embodiments, the peptide is having a sequence selected from SEQ ID NOS: 1-24, an analog or a fragment thereof.

In one or more embodiments, the peptide is having a sequence selected from SEQ ID NO: 1 (GYYWSWIRQPPGKGEEWIG), SEQ ID NO: 2 (pCONS-FIEWNKLRFRQGLEW), and SEQ ID NO: 13 (HGYYWSWIRQPPGKGLEWI), an analog or a fragment thereof.

In one or more embodiments, the microbe-based drug delivery system is devoid of at least part of the genetic material belonging to said microbe, so that the organism synthesizing the payload is no longer with active endogenous DNA (e.g, by radiation or any other method). In one or more embodiments, the microbe-based drug delivery system is devoid of the entire genetic material belonging to said microbe. In one or more embodiments, the microbe-based drug delivery system is devoid of replication genes, pathogenicity genes, and/or accessory genes. Each possibility represents a separate embodiment of the invention.

In one or more embodiments, the microbe-based drug delivery system is devoid of the entire genetic material belonging to said microbe.

In one or more embodiments, the microbe is nonpathogenic to a mammal (e.g. as in the case of measles virus as a vaccine carrier, and others).

In one or more embodiments, the microbe is selected from a virus, a bacteriophage, a fungi, and/or a bacterium.

In one or more embodiments, the peptide is coupled to a surface protein of the microbe or microbe like particle.

In one or more embodiments, the peptide is coupled to a surface protein of the microbe or microbe like particle indirectly via a linker or directly. In one or more embodiments, the peptide is coupled to a surface protein of the microbe or microbe like particle indirectly via a linker. In one or more embodiments, the peptide is coupled to a surface protein of the microbe or microbe like particle directly, via a peptide bond.

In one or more embodiments, the microbe is a microbe which is non-pathogenic to mammals, in particular to humans.

In one or more embodiments, the microbe is a virus or virus like particle and wherein the peptide is coupled to a coat protein of said virus or virus like particle.

In one or more embodiments, the microbe is a virus.

In one or more embodiments, the virus is a plant virus.

In one or more embodiments, the plant virus is selected from a group consisting of: tobacco mosaic virus (TMV), cowpea mosaic virus (CPMV), cowpea chlorotic mottle virus (CCMV), physalis mottle virus (PhMV), brome mosaic virus, red clover necrotic mosaic virus (RCNMV), hibiscus chlorotic virus, and potato virus X (PVX).

In one or more embodiments, the plant virus is a tobacco mosaic virus (TMV).

In one or more embodiments, the virus is a mammalian virus selected from a group consisting of: Hepatitis B Virus (HBV), Human Immunodeficiency Virus (HIV), Human Papillomavirus (HPV), Coxsackievirus A16 (CA16), a Poliovirus Virus-Like particle, and Enterovirus 71 (EV71).

In one or more embodiments, the microbe or microbe like particle comprises an expression vector comprising a nucleic acid sequence encoding a peptide selected from SEQ ID NOS: 1-24, an analog or a fragment thereof.

In one or more embodiments, the peptide is having at least 80% sequence identity to SEQ ID NOS: 1-24.

In one or more embodiments, the peptide is having at least 90% sequence identity to SEQ ID NOS: 1-24.

The present invention further provides a pharmaceutical composition comprising a microbe-based drug delivery system as herein disclosed, and a pharmaceutically acceptable carrier. It should be construed that the microbe-based drug delivery system includes a population of microbes. The microbe based delivery system may comprise a microbe population wherein each microbe exhibits the same peptide selected from SEQ ID NOS: 1-24, an analog or a fragment thereof. In some embodiments, the pharmaceutical composition comprises several populations of microbes, wherein each population exhibits a different peptide selected from SEQ ID NOS: 1-24, an analog or a fragment thereof. In some embodiments, the pharmaceutical composition comprises microbes each of which exhibits more than one peptide selected from SEQ ID NOS: 1-24, an analog or a fragment thereof.

The present invention further provides a method of introducing into a cell a peptide having an SLE therapeutic efficacy, the method comprising contacting the cell with the pharmaceutical composition as herein disclosed.

The present invention further provides a method of delivering into a subject a peptide having an SLE therapeutic efficacy, the method comprising administering to the subject the pharmaceutical composition as herein disclosed.

The present invention further provides a pharmaceutical composition as herein disclosed for use in the treatment of systemic lupus erythematosus (SLE).

The present invention further provides a method of treating systemic lupus erythematosus (SLE) and/or a disease or symptoms associated with SLE, the method comprises administering to a subject in need thereof an effective amount of the pharmaceutical composition as herein disclosed.

In one or more embodiments, the subject is a human subject.

In one or more embodiments, the pharmaceutical composition is administered to the subject via an intravenous, a subcutaneous, intradermal, or an oral administration. Each possibility represents a separate embodiment of the invention.

The present invention further provides an expression vector comprising a nucleic acid sequence encoding a sequence selected from SEQ ID NOS: 1-24, an analog or a fragment thereof, and one or more portion or moiety of a microbe or microbe like particle.

### BRIEF DESCRIPTION OF THE DRAWINGS

For better understanding of the present invention and in order to exemplify how it may be implemented in practice, several embodiments are hereby described, which should be interpreted only as non-limiting examples, with reference to the accompanying figures. It is noted that the sizes and scale of the embodiments presented in the figures are exemplary and non-limiting.

FIG. 1 is a flow chart depicting the various steps of a method for producing the microbe or microbe like particle comprising a peptide having an SLE therapeutic efficacy, according to some embodiments.

### DESCRIPTION OF SPECIFIC EMBODIMENTS OF THE INVENTION

Although the invention is illustrated and described herein as embodied in examples provided herein, the invention is not limited to the details shown because various modifications and changes may be made without departing from the invention and the equivalents of the claims. However, the construction and method of operation of the invention together with additional objects and advantages thereof will be best understood from the following description of specific embodiments when read in connection with the accompanying figures.

The present invention pertains to novel compositions and to methods of treating SLE. Specifically, the invention pertains to microbe-based drug delivery systems of therapeutic SLE peptides.

Among the attributes of the present invention are at least one of the following: i) an efficacious results of said peptides in treating SLE, ii) stability in the blood circulation following administration into the body, iii) an effective affinity to target(s) within the body, iv) an effective production via scalable fermentation or molecular farming, and v) safety—the use of nonpathogenic, non-replicable, biocompatible and biodegradable particles.

In one or more embodiments, various peptides efficacious in treating SLE are herewith applicable for use in microbe-based drug delivery systems.

As used herein the term “a peptide having a therapeutic SLE efficacy” refers to any peptide/protein/polypeptide that entails a therapeutic efficacy in treating SLE.

As used herein the term “a therapeutic SLE efficacy” refers to a peptide/polypeptide/protein which when administered to a subject afflicted with an SLE effects beneficial or desired results in the treatment of SLE. Monitored beneficial results may include reduction in the levels of anti-dsDNA autoantibodies, ameliorating kidney disease, as indicated by a decrease in proteinuria and in the intensity of immune-complex deposits in the Kidneys, or any change in any of the disease activity indices. Exemplary suitable indices that are known in the art include, without limitation, Systemic Lupus Erythematosus Disease Activity Index (SLEDAI), The British Isles Lupus Assessment Group (BILAG), Systemic Lupus Erythematosus Responder Index (SRI), and quality of life (QoL) indices.

In one non limiting example, the peptide is having a sequence as set forth in SEQ ID NO: 1-GYYWSWIRQPPGKGEEWIG (a.k.a. Edratide). It has been found previously as illustrated in International Patent Application Publication No. WO 2002/067848, the content of which is incorporated herein by reference, that peptides based on the CDR of the human monoclonal anti-DNA 16/6Id antibody are capable of immunomodulating SLE-associated responses. Such peptides were tested and shown to inhibit lymph node cell proliferation in an SLE mice model, to inhibit the proliferative response of peripheral blood lymphocytes (PBL) of SLE patients to the human anti-DNA 16/6Id mAb, and to ameliorate disease manifestations of mice afflicted with spontaneous or experimental SLE.

Thus, the present invention encompasses, in one embodiment, synthetic peptides comprising a sequence consisting of, or found within, a CDR of the pathogenic human monoclonal anti-DNA 16/6Id antibody for use in the microbe-based drug delivery systems of the invention. The synthetic peptides may be derived from a CDR found in the heavy chain of the human 16/6Id mAb. The CDR regions of the heavy chains of the human 16/6Id mAb have the sequences substantially as denoted by SEQ ID NO: 3, SEQ ID NO: 4, or SEQ ID NO: 24 as follows:

CDR1
[SEQ ID NO: 3]
FSGYYWS
CDR2
[SEQ ID NO: 24]
EINHSGSTNYKTSLKS
CDR3
[SEQ ID NO: 4]
GLLRGGWNDVDYYYGMDV

The heavy chain CDR (hCDR) peptides of the invention contain at least 12 and at most 30 amino acid residues and may comprise, a sequence identical to a sequence selected from the group consisting of SEQ ID NO: 3, 4 and 24, or a sequence found within said SEQ ID NO: 3, 4 or 24, or a sequence obtained by: (i) replacement of one or more, in some embodiments replacement of one, in some embodiments replacement of two, in some embodiments replacement of three of the amino acid residues of the SEQ ID NO: 3, 4 and 24 sequences by different amino acid residues; (ii) deletion of one or more amino acid residues from the SEQ ID NO: 3, 4 and 24 sequences; or (iii) addition of one or more amino acid residues to the SEQ ID NO: 3, 4 and 24 sequences.

The hCDR peptides of the invention, besides the hCDR sequence, contain further amino acid residues, in some embodiments amino acid residues of the sequences of the human 16/6Id mAb flanking the hCDR sequences or sequences obtained by replacement of one or more of the amino acid residues of the hCDR flanking sequences by different amino acid residues, by deletion of one or more amino acid residues from the hCDR flanking sequences or by addition of one or more amino acid residues to the hCDR flanking sequences.

In one embodiment of the invention, a peptide based on the CDR1 of the heavy chain of the human 16/6Id mAb is a peptide of the sequence substantially as denoted by SEQ ID NO: 5

[SEQ ID NO: 5]
X₁ Y Y W S W I X₂ Q X₃ P X₄ X₅ G X₆ E W I G

wherein X₁ is Gor TG; X₂ is R or K; X₃ is P or S; X₄ is G or E; X₅ is K or D; and X₆ is E, L or S.

In another embodiment, the peptide of SEQ ID NO:1 is a 19-mer peptide having the sequence: GYYWSWIRQPPGKGEEWIG [SEQ ID NO:1]

Also encompassed are analogs of SEQ ID NO: 1 obtained by replacement and/or addition and/or deletion of amino acid residues of SEQ ID NO:1. Applicable examples include:

[SEQ ID NO: 6]
GYYWSWIRQPPGKGLEWIG
[SEQ ID NO: 7]
GYYWSWIRQPPGKGSEWIG
[SEQ ID NO: 8]
GYYWSWIRQPPGDGEEWIG
[SEQ ID NO: 9]
GYYWSWIKQPPGKGEEWIG
[SEQ ID NO: 10]
GYYWSWIRQSPGKGEEWIG
[SEQ ID NO: 11]
GYYWSWIRQPPEKGEEWIG
[SEQ ID NO: 12]
TGYYWSWIRQPPGKGEEWIG
[SEQ ID NO: 13]
HGYYWSWIRQPPGKGLEWI

In a further embodiment, a peptide based on the CDR3 of the heavy chain of the human 16/6Id mAb, is a peptide of the sequence substantially as denoted by SEQ ID NO: 14:

[SEQ ID NO: 14]
Y Y C A R X₁ L L X₂ X₃ X₄ X₅ X₆ D V D Y X₇ G X₈ D V

wherein Xₜ is G or F; X₂ is R or A; X₃ is Gor A; X₄ is G or A; X₅ is W or A; X₆ is N or A; X₇ is Y or W; and X₈ is M or Q.

In one embodiment, the peptide of is having the sequence:

[SEQ ID NO: 15]
YYCARGLLRGGWNDVDYYGMDV

Also encompassed are analogs of SEQ ID NO: 15 obtained by replacement and/or addition and/or deletion of amino acid residues of SEQ ID NO:15. Applicable examples include:

[SEQ ID NO: 16]
YYCARGLLRGGWADVDYYGMDV
[SEQ ID NO: 17]
YYCARGLLRGGADVDYYGMDV
[SEQ ID NO: 18]
YYCARGLLRGAWNDVDYYGMDV
[SEQ ID NO: 19]
YYCARGLLRAGWNDVDYYGMDV
[SEQ ID NO: 20]
YYCARGLLAGGWNDVDYYGMDV
[SEQ ID NO: 21]
YYCARFLLRGGWNDVDYYGMDV
[SEQ ID NO: 22]
YYCARGLLRGGWNDVDYYGQDV
[SEQ ID NO: 23]
YYCARGLLRGGWNDVDYWGMDV.

Applicable fragments of a peptide derived from SEQ ID NOs: 1-23 are ones that contain at least 12 amino acids derived from SEQ ID NOs: 1-23. In some embodiments, the peptide comprises or consists of 12 to 19 amino acids derived from the amino acid sequence: SEQ ID NOs: 1-23. In some embodiments, the peptide comprises or consists of 12 to 18, 12 to 17, 12 to 16, 12 to 15, or 12 to 14 amino acids derived from the amino acid sequence: SEQ ID NOs: 1-23. Each possibility represents a separate embodiment of the invention.

In another non limiting example, the peptide has a sequence as set forth in SEQ ID NO: 2-FIEWNKLRFRQGLEW (a.k.a. pCONS). It has been found previously as illustrated in Bevra H. Hahn et al. Arthritis & Rheumatism, Vol. 44, No. 2, February 2001, pp 432-441, that the peptide of SEQ ID NO: 2 is capable of delaying the appearance of autoantibodies and prolong survival in a mouse model of SLE.

As used herein, the term “peptide” indicates a sequence of amino acids linked to each other by peptide bonds. Peptides according to some embodiments of the present invention consist of 12-19 amino acids, for example 12-15 amino acids.

As used herein, the herein peptide of SEQ ID NOS: 1-24 comprises a peptide of at least 12 amino acids (aa), at least 13 aa, at least 14 aa, at least 15 aa, at least 16 ss, at least 17 aa, at least 18 aa, or at least 19 aa. Each possibility represents a separate embodiment of the invention. In some embodiments, the peptide comprises 12 to 18, 12 to 17, 12 to 16, 12 to 15, 12 to 14, 13 to 19, 13 to 18, 13 to 17, 13 to 16, 13 to 15, or 13 to 14 amino acids. Each possibility represents a separate embodiment of the invention. In some embodiments, a peptide according to the present invention is up to 19 amino acids, for example up to 18 aa, 17 aa, 16 aa, 15 aa, 14 aa, 13 aa, 12 aa, 11 aa, or 10 aa. Each possibility represents a separate embodiment of the invention.

The term “amino acid” refers to compounds, which have an amino group and a carboxylic acid group. α-Amino acids are most abundantly used, and include the 20 natural amino acids (which are also referred to as L-amino acids except for glycine) which are found in proteins, the corresponding D-amino acids, the corresponding N-methyl amino acids, side chain modified amino acids, the biosynthetically available amino acids which are not found in proteins (e.g., 4-hydroxy-proline, 5-hydroxy-lysine, citrulline, ornithine (Orn), canavanine, djenkolic acid, β-cyanoalanine), and synthetically derived a-amino acids, such as aminoisobutyric acid, norleucine (Nle), norvaline (NorVal, Nva), homocysteine and homoserine. β-Alanine and γ-amino butyric acid are examples of 1,3 and 1,4-amino acids, respectively, and many others as well known to the art.

The terms “peptide”, and “protein” as used herein encompass native peptides, peptidomimetics (typically including non-peptide bonds or other synthetic modifications) and the peptide analogs peptoids and semi-peptoids or any combination thereof. In another embodiment, the terms “peptide”, and “protein” may apply to amino acid polymers in which at least one amino acid residue is an artificial chemical analog of a corresponding naturally occurring amino acid.

One of skill in the art will recognize that individual substitutions, deletions or additions to a peptide, or protein sequence which alters, adds or deletes a single amino acid or a small percentage of amino acids in the encoded sequence is a conservatively modified variant where the alteration results in the substitution of an amino acid with a similar charge, size, and/or hydrophobicity characteristics, such as, for example, substitution of a glutamic acid (E) to an aspartic acid (D).

Analogs and derivatives of the peptides are also within the scope of the present application.

“Derivatives” of the peptides of the invention as used herein cover derivatives which may be prepared from the functional groups which occur as side chains on the residues or the N- or C-terminal groups, by means known in the art, and are included in the invention as long as they remain pharmaceutically acceptable, i.e., they do not destroy the activity of the peptide, do not confer toxic properties on compositions containing it, and do not adversely affect the immunogenic properties thereof.

These derivatives may include, for example, aliphatic esters of the carboxyl groups, amides of the carboxyl groups produced by reaction with ammonia or with primary or secondary amines, N-acyl derivatives of free amino groups of the amino acid residues, e.g., N-acetyl, formed by reaction with acyl moieties (e.g., alkanoyl or carbocyclic aroyl groups), or O-acyl derivatives of free hydroxyl group (e.g., that of seryl or threonyl residues) formed by reaction with acyl moieties.

“Analogs” of the peptides of the invention as used herein cover compounds which have the amino acid sequence according to the invention except for one or more amino acid changes, typically, conservative amino acid substitutions. In some embodiments, an analog has at least about 75% identity to the sequence of the peptide of the invention, for example at least about 80%, at least about 85%, at least about 90%, at least about 95%, or at least about 99% identity to the sequence of the peptide of the invention.

Conservative substitutions of amino acids as known to those skilled in the art are within the scope of the present invention. Conservative amino acid substitutions include replacement of one amino acid with another having the same type of functional group or side chain e.g., aliphatic, aromatic, positively charged, negatively charged.

Conservative substitution tables providing functionally similar amino acids are well known in the art.

The following six groups each contain amino acids that are conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K), Histidine (H);
5) Isoleucine (I), Leucine (L), Methionine (M), Valine (V); and
6) Phenylalanine (F), Tyrosine (Y), Tryptophan (W).

Analogs according to the present invention may comprise also peptidomimetics. “Peptidomimetic” means that a peptide according to the invention is modified in such a way that it includes at least one non-coded residue or non-peptide bond. Such modifications include, e.g., alkylation and more specific methylation of one or more residues, insertion of or replacement of natural amino acid by non-natural amino acids, replacement of an amide bond with another covalent bond. A peptidomimetic according to the present invention may optionally comprise at least one bond which is an amide replacement bond such as urea bond, carbamate bond, sulfonamide bond, hydrazine bond, or any other covalent bond. The design of appropriate analogs may be computer assisted.

The herein peptides are to be administered with a microbe-based particle. Therapeutic peptides may be more active when presented on the surface of a microbial particle. Namely, immune determinants responses of the SLE patient may be markedly enhanced when the therapeutic peptide is presented on the surface of the microbe. Without being bound by any theory or mechanism of action, the herein peptides are therapeutically active when they interact with cells of the immune system at the immediate site of injection (e.g., SC injection and T-Cells and APC). The peptide-educated immune cells convey signals to other cells that lead to the decrease in the auto-immune response signals.

As used herein the term “microbe” is interchangeable with the term “microbe like particle” and refers to any vehicle that is derived from a microbe and is capable of displaying the herein peptide(s) having a therapeutic SLE efficacy on its surface. As used herein a “microbe-based drug delivery system” may be an entity which contains all or most of the components of a microbe but is modified to further include, for example, a peptide(s) having a therapeutic SLE efficacy.

In one or more embodiments, the microbe is a microbe that is non-pathogenic to mammals or inert in mammals, i.e., it does not inflict a medical condition or disease on a mammal, in particular, a human. In one or more embodiments, the microbe is a virus which is non-pathogenic to humans.

In one embodiment, the microbe is selected from a virus, a bacteriophage, a fungus, and/or a bacterium. The microbe may be devoid of genetic material. The microbe may be devoid of genetic material belonging to the microbe and may include other external genetic material. In another embodiment, the microbe is nonpathogenic to a mammal. In one or more embodiments, the microbe is selected from a virus, a bacteriophage, a fungus, and/or a bacterium. In one or more embodiments, the peptide is coupled to a surface protein of the microbe or microbe like particle. In one or more embodiments, the peptide is displayed on the surface of said microbe or microbe like particle.

The term “displayed on the surface” means that the peptide on the surface may be recognizable to an immune system particle in vivo or in vitro. The peptide may be exposed to the extracellular environment of the microbe. The peptide may be coupled to the surface of the microbe such that it is exposed to the surrounding environment.

The virus may be a virus like particle (VLP), i.e., an assembly of coat proteins of viruses, or a viral nanoparticle (VNP). VLPs are genome free and are considered noninfectious. Thus, in one or more embodiments, the virus is nonpathogenic to a mammal. As used herein the term “nonpathogenic” refers to any virus that does not cause disease in its host organism. Nonpathogenic viruses are characterized by their inability to produce pathogenic effects, thereby rendering them safe for various applications. The nonpathogenic nature of the virus ensures that it does not induce harmful immune responses or clinical symptoms in the host. In exemplary embodiments, when using viruses as vehicles for drugs, at least part of the viral genome can be removed. Such parts may include genes responsible for replication and pathogenicity. In one or more embodiments, the microbe-based drug delivery system is devoid of genes necessary for viral replication to prevent the virus from multiplying uncontrollably within the host (i.e. replication genes). In one or more embodiments, the microbe-based drug delivery system is devoid of genes that contribute to the virus's ability to cause disease (i.e. pathogenicity genes). This includes genes involved in virulence, which may trigger harmful immune responses or toxic effects in the host. In one or more embodiments, the microbe-based drug delivery system is devoid of genes that are not essential for the therapeutic purpose but may contribute to the virus's ability to evade the immune system or cause unwanted side effects (i.e. accessory genes). Each possibility represents a separate embodiment of the invention.

In an alternative embodiment, genes of the virus can be modified to produce a nonpathogenic virus. For example, when Tobacco Mosaic Virus (TMV) is used some genes can be removed or modified. For example, replication genes that can be modified or removed include RNA-dependent RNA polymerase (RdRp), methyltransferase, and helicase domains, Movement Protein (MP) genes, and/or Coat Protein (CP) genes. These modifications ensure that the TMV vector is safe and effective for delivering therapeutic agents or expressing recombinant proteins without causing significant disease symptoms or uncontrolled spread.

In one or more embodiments, the virus is noninfectious to a mammal. In one or more embodiments, the herein peptide is coupled to a surface protein of the virus. In one or more embodiments, the peptide is displayed on the surface of virus and is coupled to a coat protein of the virus. In one or more embodiments, the peptide is displayed on the surface of virus and is coupled to a capsid protein of the virus.

The virus may be a plant or a mammalian virus. Plant viruses are viruses which may induce a viral plant disease. A mammalian virus is a virus which may induce a viral disease in a mammal. Various plant viruses are contemplated including, without limitation, a tobacco mosaic virus (TMV), cowpea mosaic virus (CPMV), cowpea chlorotic mottle virus (CCMV), physalis mottle virus (PhMV), brome mosaic virus, red clover necrotic mosaic virus (RCNMV), hibiscus chlorotic virus, and potato virus X (PVX). In one embodiment, the plant virus is a tobacco mosaic virus (TMV). Each possibility represents a separate embodiment of the invention.

Tobacco mosaic virus (TMV) is a positive-sense single-stranded RNA virus species in the genus Tobamovirus that infects a wide range of plants, especially tobacco and other members of the family Solanaceae. Tobacco mosaic virus (TMV) is a rod-shaped particle, 300 nm long, consisting of 2130 identical protein submits of 17,500 MW arranged as a helix around an RNA molecule of 2×10⁶ MW. The coat protein (TMVP) of the virus contains 158 amino acid residues.

Various mammalian viruses are contemplated including, without limitation, Hepatitis B Virus (HBV), Human Immunodeficiency Virus (HIV), Human Papillomavirus (HPV), Coxsackievirus A16 (CA16), a Poliovirus Virus-Like particle, and Enterovirus 71 (EV71). Each possibility represents a separate embodiment of the invention.

Exemplary applicable bacteriophages include, for example, MS2, P22, Qβ and M13. Each possibility represents a separate embodiment of the invention.

The herein peptides may be incorporated to a surface protein of the microbe by conjugation. Covalent attachments make use of reactive amino acid side chains of the protein structure. Functional favorable groups are principally amines (Lys), carboxylates (Asp, Glu), thiols (Cys), and aromatic groups (Tyr, Trp), all of which can be utilized for bioconjugation chemistries. Some powerful chemical reactions used for bioconjugation on these moieties include NHS esters, carbodiimides, maleimides, and click chemistries respectively. Moreover, residues such as lysine can be site-specifically engineered into the capsid protein to provide non-native reactive sites.

The surface attached peptides may be either released in a controllable manner, e.g., through dedicated enzymes, e.g., Cathepsin B. Alternatively, the peptides may be linked in a stable manner and mediate their therapeutic effect when conjugated to the microbe.

### Production of Microbe or Microbe Like Particles

The herein particles may be produced in a cell-based system (e.g., using an Escherichia coli or Pseudomonas aeruginosa), or in a cell free system.

A peptide of the invention may be incorporated onto the microbe surface protein by a chemical conjugation process. Microbe particles are mixed with peptides and the peptides may be individually chemically conjugated to the surface of the microbes to produce distinct populations of microbes, each displaying a unique peptide.

The described invention exploits the unique properties of the tobacco mosaic virus (TMV) that is amenable to such chemical conjugation.

The conjugation to a surface protein of the microbe may be a direct conjugation or an indirect conjugation.

As used herein, the term “direct conjugation” refers to cases wherein the peptide of the invention is bound/conjugated to the surface/coat protein without any moiety that links the peptide to the surface/coat protein.

As used herein, the term “indirectly” refers to cases wherein each of the peptide of the invention and the surface/coat protein are bound to a linker or a spacing element and not directly to one another. In some embodiments, the peptide is covalently bound to the linker. In some embodiments, the peptide is non-covalently bound to the linker.

The term “linker” refers to a molecule or macromolecule serving to connect different moieties, that is the peptide of the invention and a surface protein of a microbe. In one embodiment, a linker may also facilitate other functions, including, but not limited to, preserving biological activity, maintaining sub-units and domains interactions, and others. The linker may have one or more amino acids or a chemical moiety.

As used herein, the term “covalent bond” refers to any bond which comprises or involves electron sharing. Non-limiting examples of a covalent bond include, but are not limited to: peptide bond, glyosidic bond, ester bond, phosphor diester bond.

As used herein, the term “non-covalent bond” encompasses any bond or interaction between two or more moieties which do not comprise or do not involve electron sharing. Non-limiting examples of a non-covalent bond or interaction include, but are not limited to, electrostatic, p-effect, van der Waals force, hydrogen bonding, and hydrophobic effect.

Further optional methods include producing a library of random sequence peptide insertions created by recombinant DNA methods, the resulting plasmid population is introduced into E. coli by transformation, and a library of microbe particles is produced. Each individual particle in the population displays a different peptide on its surface and contains within it (in the form of mRNA) the genetic information for its synthesis. The population of particles representing a random sequence peptide library is incubated with a monoclonal antibody immobilized on a surface. The vast majority of particles typically fail to bind the antibody and are washed away and discarded. Any particles whose peptides exhibit binding of the antibody are then specifically eluted, the RNA they contain is copied into DNA by reverse transcription, amplified by PCR and then re-cloned into an expression plasmid (e.g., pDSP62 or pDSP62 (am)) for production of the affinity-selected particles. Selection is typically conducted iteratively (i.e., more than twice), and in some embodiments, for 3 to 5 rounds.

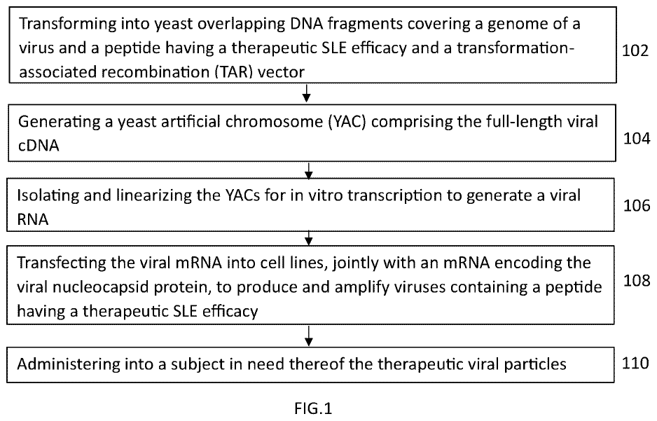

One further exemplary method based on a yeast synthetic genomics platform is elaborated in Thao, T. T. N. et al. Rapid reconstruction of SARS-COV-2 using a synthetic genomics platform (Nature volume 582, pages 561-565 (2020)). This method is briefly illustrated in the flow chart of FIG. 1. The transformed DNA should generate virus particles that exhibit the herein peptide of interest on their surface. In step 102 yeast cells are transformed with overlapping DNA fragments including i) a viral genome; ii) a peptide of interest having a therapeutic SLE efficacy; and iii) a transformation associated recombination (TAR) vector. The DNA fragments encoding the peptide of interest having a therapeutic SLE efficacy may be inserted in a region of the viral encoding a portion or moiety of the virus which is exposed to the environment, e.g. on the surface of the virus. Transformed DNA fragments are assembled by homologous recombination in yeast to generate in step 104 a yeast artificial bacteria (YAC) that contains the full-length viral cDNA sequence. In vitro production of infectious capped viral RNA starts with the isolation of the YAC, followed by plasmid linearization to provide a DNA template for run-off T7 RNA polymerase-based transcription (step 106). Virus rescue is initiated by electroporation of BHK-MHV-N cells, after which virus production and amplification is carried out by culturing the virus with susceptible cells (step 108).

Recovered viruses may be used for administration in vivo or into production for administering to subjects.

The present invention further provides an expression vector comprising a nucleic acid sequence encoding a peptide having an SLE therapeutic efficacy, an analog or a fragment thereof, and one or more portion or moiety of a microbe or microbe like particle The nucleic acid sequence encoding a peptide having an SLE therapeutic efficacy may be inserted into a region of a nucleic acid sequence encoding a portion or moiety of the microbe or microbe like particle that is exposed to the environment such as envelope proteins of a virus in case that the microbe is a virus.

In one or more embodiments, the peptide having an SLE therapeutic efficacy is selected from SEQ ID NOS: 1-24. In one or more embodiments, the one or more portion or moiety of a microbe or microbe like particle is a coat protein, e.g., a capsid protein of a microbe.

### Peptide Synthesis

According to one embodiment, the peptide of the invention (SEQ ID NOS 1-24) may be synthesized or prepared and thereafter attached onto the surface of microbe particles. According to one embodiment, the peptide of the invention (SEQ ID NOS 1-24) may be synthesized or prepared by any method and/or technique known in the art for peptide synthesis. According to another embodiment, the peptide may be synthesized by a solid phase peptide synthesis method of Merrifield (see J. Am. Chem. Soc, 85:2149, 1964). According to another embodiment, the peptide of the invention can be synthesized using standard solution methods, which are well known in the art (see, for example, Bodanszky, M., Principles of Peptide Synthesis, Springer-Verlag, 1984).

The synthesized peptides (SEQ ID NOS 1-24) may be chemically attached to the surfaces of virus particles via peptide bonds via covalent linkage between the peptide and the viral coat proteins. Various methods for such chemical attachments are contemplated. For example, Carbodiimide-Mediated Coupling may be utilized using carbodiimides, such as EDC (1-Ethyl-3-(3-dimethylaminopropyl) carbodiimide), to form peptide bonds between carboxyl groups on the viral surface and amino groups on the peptide. Further exemplary methods of attaching the peptide onto the surface of a virus include Thiol-Maleimide Conjugation (Indirect Peptide Bond Formation), and Sortase-Mediated Ligation, Native Chemical Ligation (NCL).

### Formulation of the Composition and Methods of Treating SLE

The invention further relates to pharmaceutical compositions comprising microbes or microbe-based particles comprising the herein peptide(s) having an SLE therapeutic efficacy according to the invention, optionally with a pharmaceutically acceptable carrier. In one embodiment, the pharmaceutical compositions comprise microbes or microbe-based particles comprising at least one peptide of the invention, such as a peptide having a sequence selected from SEQ ID NO: 1-24.

In yet another aspect the invention provides a method of introducing into a cell a peptide having a sequence selected from SEQ ID NO; and SEQ ID NO: 2, an analog or fragment thereof, or a nucleic acid sequence encoding said peptides, an analog or a fragment thereof, the method comprising contacting the cell with the pharmaceutical composition as herein disclosed.

In yet another aspect the invention provides a method of delivering into a subject a peptide having a sequence selected from SEQ ID NO: 1 and SEQ ID NO: 2, an analog or a fragment thereof, or a nucleic acid sequence encoding said peptide, an analog or a fragment thereof, the method comprising administering to the subject the pharmaceutical composition as herein disclosed.

In one or more embodiments, the herein pharmaceutical compositions are for use in the treatment of systemic lupus erythematosus (SLE).

In yet another aspect the invention provides a method of treating systemic lupus erythematosus (SLE) and/or a disease or symptoms associated with SLE, the method comprising administering to a subject in need thereof an effective amount of the pharmaceutical composition as herein disclosed.

The composition can be formulated as granules, powder, capsules, tablet, film, emulsion, lozenge, tincture, sachets, a hard or soft gelatin capsule or a suspension. Exemplary diluents are water and alcohols, for example, ethanol, benzyl alcohol and polyethylene alcohols, either with or without the addition of a pharmaceutically acceptable surfactant, suspending agent, or emulsifying agent. Pharmaceutically compatible binding agents, and/or adjuvant materials can be included as part of the composition. Capsules may contain surfactants, lubricants, and inert fillers, such as lactose, sucrose, calcium phosphate, and corn starch. Tablet forms may include one or more of lactose, sucrose, mannitol, corn starch, potato starch, alginic acid, microcrystalline cellulose, acacia, gelatin, guar gum, colloidal silicon dioxide, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenges can comprise the active ingredient in a flavor, usually sucrose and acacia or tragacanth, as well as pastilles comprising the active formulation in an inert base, such as gelatin and glycerin, or sucrose and acacia, emulsions, gels, and the like containing, in addition to the active formulation, such carriers as are known in the art.

The term “pharmaceutically acceptable carrier” applied to pharmaceutical compositions of the invention refers to a diluent, excipient, or vehicle with which the herein microbe or microbe like particle are administered. The carrier refers to an excipient that is useful in preparing a pharmaceutical composition that is generally safe, non-toxic, and neither biologically nor otherwise undesirable, and includes excipients that are acceptable for veterinary use as well as human pharmaceutical use.

Carriers of the present invention may include: (1) excipients or formularies that transport, but do not specifically target a molecule to a cell (referred to herein as non-targeting carriers); and (2) excipients or formularies that deliver a molecule to a specific site in a subject or a specific cell (i.e., targeting carriers). Examples of non-targeting carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Any suitable route of administration is encompassed by the invention, including oral, intravenous, subcutaneous, intraarticular, intramuscular, inhalation, intranasal, intrathecal, intraperitoneal, intradermal, transdermal or other known routes. In some embodiments, the pharmaceutical composition of the invention is administered by an intravenous, a subcutaneous, intradermal, or an oral administration. Each possibility represents a separate embodiment of the invention.

The dose ranges for the administration of the compositions of the present invention should be large enough to produce the desired effect, whereby, for example, an immune response to the SLE-inducing autoantibody, as measured by T cell proliferation in vitro, is substantially prevented or inhibited, and further, where the disease is significantly treated. The doses should not be so large as to cause adverse side effects, such as unwanted cross reactions, generalized immunosuppression, anaphylactic reactions and the like.

Effective doses of the peptides of this invention for use in treating SLE are in the range of about 1 μg to 1 mg and up to 100 mg/kg body weight.

The herein nanoparticles of the invention are aimed at inhibiting or suppressing specific antigen responses of SLE patients, without affecting all other immune responses. This approach is of the utmost importance since most diagnosed patients are young women that have to be treated for many years and the currently accepted treatment for SLE involves administration of immunosuppressive agents, such as corticosteroids and/or cytotoxic drugs, that are both non-specific and have multiple adverse side effects.

The invention further relates to a method for the treatment of systemic lupus erythematosus (SLE) comprising administering to an SLE patient an effective amount of microbes or microbe-based particles comprising the herein peptide(s) having an SLE therapeutic efficacy of the invention.

The invention still further relates to method of immunomodulation of SLE-associated responses in a SLE patient which comprises administering to said SLE patient an effective amount of an effective amount of a microbes or microbe-based particles comprising the herein peptide(s) having an SLE therapeutic efficacy of the invention.

In one embodiment, the method comprises down-regulating the levels of matrix metalloproteinase (MMP)-3 and/or MMP-9 activities in a SLE patient. In another embodiment, the method comprises immunomodulating the level of a cytokine activity in a SLE patient, particularly down-regulating the level of IL-2 and/or IFN-γ activity and/or up-regulating the level of TGF-β activity, in a SLE patient.

The invention still further relates to the use of microbes or microbe-based particles comprising the herein peptide(s) having an SLE therapeutic efficacy of the invention for the preparation of a pharmaceutical composition, in particular for treatment of SLE, more particularly for immunomodulation of SLE-associated responses in a SLE patient such as down-regulation of MMP-3 and/or MMP-9 and/or IL-2 and/or IFN-γ or up-regulation of TGF-β levels in a SLE-patient.

The present invention will now be described in more detail in the following non-limiting Examples and the accompanying figures.

### Definitions

The term “consisting essentially of” means that the composition, method or structure may include additional ingredients, steps and/or parts, but only if the additional ingredients, steps and/or parts do not materially alter the basic and novel characteristics of the claimed composition, method or structure.

The term “consisting of” means including and limited to.

As used herein the term “about” refers to ±10% or ±5%.

The terms “comprise”, “comprising”, “includes”, “including”, “having” and their conjugates mean “including but not limited to”.

The term “treating” or “treatment” of SLE in a patient refers to administration of the herein composition for preventing or ameliorating or delaying the appearance of one or more of SLE symptoms.

The term “subject” refers to a human or a non-human mammal.

The term “administration” refers to providing or giving a subject a therapeutic agent by any effective route.

The term “effective amount” or “therapeutically effective amount” refers to a quantity sufficient to, when administered to the subject, affect a beneficial or desired result, including clinical results associated with SLE.

### EXAMPLES

The invention will be described in detail by way of specific examples. The following examples are offered for illustrative purposes and are not intended to limit the invention in any manner. Those of skill in the art will readily recognize a variety of non-critical parameters, which can be changed or modified to yield essentially the same results. In one or more embodiments, the amounts in the examples should be read with the prefix “about”.

### Example 1. Preparation in Yeast Cells of Tobacco Mosaic Virus (TMV) Particles Displaying on their Surface a Peptide (SEQ ID NO:1) Having an SLE Therapeutic Efficacy

Yeast cells are transformed with overlapping DNA fragments including i) a TMV genome comprising an oligonucleotide sequence encoding SEQ ID NO: 1 in a region of the TMV genome encoding viral envelope proteins; and ii) a transformation associated recombination (TAR) vector. Transformed DNA fragments are assembled by homologous recombination in the yeast to generate a YAC that contains the full-length viral cDNA sequence. In vitro production of infectious capped viral RNA starts with the isolation of the YAC, followed by plasmid linearization to provide a DNA template for run-off T7 RNA polymerase-based transcription. TMV rescue is initiated by electroporation of BHK-MHV-N cells, after which virus production and amplification is carried out by culturing the TMV with susceptible cells. Recovered TMVs may are used for administration in vivo as follows.

### Example 2. Preparation in Plant Cells of Tobacco Mosaic Virus (TMV) Particles Displaying on their Surface a Peptide (SEQ ID NO:1) Having an SLE Therapeutic Efficacy

Plant cells (in a culture tissue or in a whole plant) are transformed with overlapping DNA fragments including i) a TMV genome comprising an oligonucleotide sequence encoding SEQ ID NO:1 in a region of the TMV genome encoding viral envelope proteins; and ii) a transformation associated recombination (TAR) vector according to procedures know in the art (e.g., see Curr. Opin. Biotechnol. 2007, 18 (2), 134-41).

### Example 3. Preparation of Bacteriophage Displaying on their Surface a Peptide (SEQ ID NO:1) Having an SLE Therapeutic Efficacy

3.1 Bacteriophage displaying on their surface a peptide (SEQ ID NO:1) having an SLE therapeutic efficacy can be prepared following mutatis mutandis a procedure disclosed in the procedure described in J. Mol. Biol. 1986, 189 (1), 113-30.
3.2 A population of a bacteriophage is transformed with overlapping DNA fragments including i) a bacteriophage genome comprising an oligonucleotide sequence encoding SEQ ID NO: 1 in a region of the bacteriophage genome encoding viral envelope proteins; and ii) a transformation associated recombination (TAR) vector. Transformed DNA fragments are assembled by homologous recombination in the yeast to generate a YAC that contains the full-length viral cDNA sequence. In vitro production of infectious capped viral RNA starts with the isolation of the YAC, followed by plasmid linearization to provide a DNA template for run-off T7 RNA polymerase-based transcription. TMV rescue is initiated by electroporation of BHK-MHV-N cells, after which virus production and amplification is carried out by culturing the TMV with susceptible bacteriophage. Recovered TMVs may are used for administration in vivo as follows.

### Example 4-Preparing a Virus Displaying the Peptide by Chemically Attaching the Peptide to the Surface of the Virus

### Carbodiimide-Mediated Coupling

Carbodiimides, such as EDC (1-Ethyl-3-(3-dimethylaminopropyl) carbodiimide), is used to form peptide bonds between carboxyl groups on the viral surface and amino groups on the peptide (SEQ ID NOS: 1-24).

The carboxyl groups on the viral coat proteins are activated using EDC in the presence of N-hydroxysuccinimide (NHS) or sulfo-NHS. This forms an NHS ester intermediate.

The peptide, containing a free amino group, is then added to the reaction. The activated carboxyl groups on the virus react with the amino groups on the peptide to form stable amide bonds. The virus particles with conjugated peptides are purified using centrifugation or chromatography to remove unreacted peptides and reagents.

### Example 5. Preparation of TMV Particles Displaying on their Surface a Peptide Having a Sequence Selected from One of SEQ ID NO:2 to SEQ ID NO: 24 Having an SLE Therapeutic Efficacy

TMV particles displaying on their surface a peptide having one of amino acid sequences (SEQ ID NO:2-24) having an SLE therapeutic efficacy are prepared mutatis mutandis according to the procedure described in Examples 1 to 4.

### Example 6. Animal Models of SLE

Various mouse models are available for in vivo studies as follows:
i) NZB/NZW F1 mice and MRL/lpr mice are prone to developing lupus (Sun et al (2004) Regulation of immune function by calorie restriction and cyclophosphamide treatment in lupus-prone NZB/NZW F1 mice. Cell Immunol. 2004 March; 228 (1): 54-65. doi: 10.1016/j.cellimm.2004.04.001. PMID: 15203320; and Zhou et al (2004) Identification of systemically expanded activated T cell clones in MRL/lpr and NZB/W F1 lupus model mice. Clin Exp Immunol. 2004 June; 136 (3): 448-55. doi: 10.1111/j.1365-2249.2004.02473.x. PMID: 15147346; PMCID: PMC1809066).
ii) Li and colleagues (Li et al (2004) Induction of systemic lupus erythematosus syndrome in BALB/c mice by immunization with active chromatin. Acta Pharmacol. Sin. 2004 June; 25 (6): 807-11. PMID: 15169636) induced a systemic lupus erythematosus syndrome in Balb/c mice by immunizing them with chromatin.
iii) Knockout of the 60 kD Ro protein, a common autoantigen in lupus and Sjögren's disease, leads to lupus in mice (Scofield (2004) a common lupus autoantigen, induces lupus. Trends Immunol. 2004 January; 25 (1): 1-3. doi: 10.1016/j.it.2003.10.014. PMID: 14698275).

The NZBxNZW method is used to test the activity in vivo of the herein microbe particles.

### The Study Includes:

(NZBxNZW) F1 mice (induced by immunization with the human 16/6 Id mAb). The affected mice will be administered with the test product (i.e., Vedratide-TMV displaying SEQ ID NO:1) in PBS, or with adjuvant, s.c. or by i.p., or i.v. injections. Edratide, i.e., SEQ ID NO: 1 is used as a control.

The treatment protocol will include an injection once a week for 10 weeks. The dose will be selected based on the molecular (mole) equivalent of Edratide used in studies, with higher and lower doses.

| | | | Route of |
|---|---|---|---|
| | | | administration (IV- |
| | | | intravenous, SC- |
| | | | subcutaneous, IP- |
| | Treatment | Vehicle | intraperitoneal) |
| 1 | Vedratide | PBS | IV |
| 2 | Vedratide | PBS + Adjuvant | IV |
| 3 | Edratide | PBS | IV |
| 4 | Edratide | PBS + Adjuvant | IV |
| 5 | Control Particle | PBS | IV |
| 6 | Control Particle | PBS + Adjuvant | IV |
| 7 | Vedratide | PBS | SC |
| 8 | Vedratide | PBS + Adjuvant | SC |
| 9 | Edratide | PBS | SC |
| 10 | Edratide | PBS + Adjuvant | SC |
| 11 | Control Particle | PBS | SC |
| 12 | Control Particle | PBS + Adjuvant | SC |
| 13 | Vedratide | PBS | IP |
| 14 | Vedratide | PBS + Adjuvant | IP |
| | Edratide | PBS | IP |
| | Edratide | PBS + Adjuvant | IP |
| 15 | Control Particle | PBS | IP |
| 16 | Control Particle | PBS + Adjuvant | IP |

The parameters monitored may include one or more of: survival, proteinuria, immune complex deposits in the kidney, and general welfare of study animals.

### Example 7

In vivo studies demonstrating the therapeutic effect of microbe-based drug delivery system comprising a TMV displaying a peptide having a systemic lupus erythematosus (SLE) of a sequence selected from one of SEQ ID NOs: 1 to 24 is performed following mutatis mutandis the procedure provided in Example 6.

### Example 8: Clinical Trials

Phase I trials for testing tolerability, safety and efficacy in SLE patients and immunological response to Vedratide can be performed on three arms of mild to moderate SLE patients, preferably with a total of at least 180 patients. The three arms may include Placebo, administration of low dose of Vedratide and administration of high dose of Vedratide (viral vehicle such as TMV displaying a peptide having an SLE of a sequence selected from one of SEQ ID NOs: 2 to 24). The treatment may take place for 26 weeks with 26 weeks extension (recruitment 6-12 months) The study design may follow Once weekly SC injection of dose 1 or 2 of Vedratide or Placebo for 52 wks:
Step #1 (wks 1-8): ad lib steroids; reach 7.5 mg or less;
Step #2 (wks 8-26): no steroid change (up to 2 wks/up to15 mg rescue);
Step #3 (wks 26-52): stable steroid dose;

The Primary End Point (PEP) in the first study will start with Safety and tolerability, and the Secondary End Points (SEP) will include efficacy and biomarker analysis. Later the PEP will become efficacy and SEP will include safety, tolerability and biomarker analyses.

The effect of Vedratide versus Placebo (and standard of care) will be evaluated based on some of the common disease activity scores, such as SLEDAI, BILAG, SRI4, or any of their derivatives or combinations.

### Main Inclusion:

1. Active SLE patients-SLEDAI>6 (ex sero) & at least one BILAG A or two B's
2. Sero+ patients—with high anti-dsDNA titers (>30IU) or ANA (>1:80)
3. On stable SLE treatment regimen and up to 20 mg prednisone

### Main Exclusion:

### Severe Active LN or Active CNS Lupus

The effect of treatment with Vedratide on gene expression can be demonstrated by monitoring the biomarkers selected from IL-1β, TNF-α, IFN-γ, IL-10, TGF-β, FoxP3, caspase, caspase 8, and BlyS in a placebo group vs. a group receiving Vedratide and a group receiving Edratide.

1. A microbe-based drug delivery system comprising a microbe or microbe like particle; and a peptide having a systemic lupus erythematosus (SLE) therapeutic efficacy, wherein said peptide is displayed on the surface of said microbe or microbe like particle.

2. The microbe-based drug delivery system according to claim 1, wherein said peptide is having a sequence selected from SEQ ID NOS: 1-24, an analog or a fragment thereof.

3. The microbe-based drug delivery system according to claim 1, wherein the peptide is having a sequence selected from SEQ ID NO: 1 (GYYWSWIRQPPGKGEEWIG), SEQ ID NO: 2 (FIEWNKLRFRQGLEW), and SEQ ID NO: 13 (HGYYWSWIRQPPGKGLEWI), an analog or a fragment thereof.

4. The microbe-based drug delivery system of claim 1, which is devoid of at least part of the genetic material belonging to said microbe.

5. The microbe-based drug delivery system of claim 1, wherein the microbe is at least one of (i) selected from a virus, a bacteriophage, a fungi, and a bacterium; and (ii) nonpathogenic to a mammal.

6. (canceled)

7. The microbe-based drug delivery system of claim 1, wherein the peptide is coupled to a surface protein of the microbe or microbe like particle.

8. The microbe-based drug delivery system of claim 1, wherein the peptide is coupled to a surface protein of the microbe or microbe like particle indirectly via a linker or directly.

9. The microbe-based drug delivery system according to claim 7, wherein the microbe is a virus or virus like particle and wherein the peptide is coupled to a coat protein of said virus or virus like particle.

10. (canceled)

11. The microbe-based drug delivery system of claim 5, wherein the virus is a plant virus.

12. The microbe-based drug delivery system of claim 11, wherein the plant virus is selected from a group consisting of: tobacco mosaic virus (TMV), cowpea mosaic virus (CPMV), cowpea chlorotic mottle virus (CCMV), physalis mottle virus (PhMV), brome mosaic virus, red clover necrotic mosaic virus (RCNMV), hibiscus chlorotic virus, and potato virus X (PVX).

13. (canceled)

14. The microbe-based drug delivery system of claim 5, wherein the virus is a mammalian virus selected from a group consisting of: Hepatitis B Virus (HBV), Human Immunodeficiency Virus (HIV), Human Papillomavirus (HPV), Coxsackievirus A16 (CA16), a Poliovirus Virus-Like particle, and Enterovirus 71 (EV71).

15. The microbe-based drug delivery system of claim 1, wherein the microbe or microbe like particle comprises an expression vector comprising a nucleic acid sequence encoding a peptide selected from SEQ ID NOS: 1-24, an analog or a fragment thereof.

16. The microbe-based drug delivery system of claim 1, wherein the peptide is having at least 80% sequence identity to SEQ ID NOS: 1-24.

17. (canceled)

18. A pharmaceutical composition comprising a plurality of microbe-based drug delivery systems of claim 1, and a pharmaceutically acceptable carrier.

19. (canceled)

20. (canceled)

21. The pharmaceutical composition of claim 18, for use in the treatment of systemic lupus erythematosus (SLE).

22. A method of treating systemic lupus erythematosus (SLE) and/or a disease or symptoms associated with SLE, the method comprises administering to a subject in need thereof an effective amount of the pharmaceutical composition according to claim 18.

23. The method of claim 22, wherein the subject is a human subject.

24. The method of claim 22, wherein the pharmaceutical composition is administered to the subject via an intravenous, a subcutaneous, intradermal, or an oral administration.

25. An expression vector comprising a nucleic acid sequence encoding a sequence selected from SEQ ID NOS: 1-24, an analog or a fragment thereof, and one or more portion or moiety of a microbe or microbe like particle.


SEQUENCE LISTING (24 sequences)

SEQ ID NO: 1 - protein, 19 aa, synthetic construct
GYYWSWIRQPPGKGEEWIG

SEQ ID NO: 2 - protein, 15 aa, synthetic construct
FIEWNKLRFRQGLEW

SEQ ID NO: 3 - protein, 7 aa, synthetic construct
FSGYYWS

SEQ ID NO: 4 - protein, 18 aa, synthetic construct
GLLRGGWNDVDYYYGMDV

SEQ ID NO: 5 - protein, 19 aa, synthetic construct
  VAR_SEQ 1: note = X is G or TG
  VAR_SEQ 8: note = X is R or K
  VAR_SEQ 10: note = X is P or S
  VAR_SEQ 12: note = X is G or E
  VAR_SEQ 13: note = X is K or D
  VAR_SEQ 15: note = X is E, L or S
XYYWSWIXQXPXXGXEWIG

SEQ ID NO: 6 - protein, 19 aa, synthetic construct
GYYWSWIRQPPGKGLEWIG

SEQ ID NO: 7 - protein, 19 aa, synthetic construct
GYYWSWIRQPPGKGSEWIG

SEQ ID NO: 8 - protein, 19 aa, synthetic construct
GYYWSWIRQPPGDGEEWIG

SEQ ID NO: 9 - protein, 19 aa, synthetic construct
GYYWSWIKQPPGKGEEWIG

SEQ ID NO: 10 - protein, 19 aa, unidentified
GYYWSWIRQSPGKGEEWIG

SEQ ID NO: 11 - protein, 19 aa, synthetic construct
GYYWSWIRQPPEKGEEWIG

SEQ ID NO: 12 - protein, 20 aa, synthetic construct
TGYYWSWIRQPPGKGEEWIG

SEQ ID NO: 13 - protein, 19 aa, synthetic construct
HGYYWSWIRQPPGKGLEWI

SEQ ID NO: 14 - protein, 21 aa, synthetic construct
  VAR_SEQ 6: note = X is G or F
  VAR_SEQ 9: note = X is R or A
  VAR_SEQ 10: note = X is G or A
  VAR_SEQ 11: note = X is G or A
  VAR_SEQ 12: note = X is W or A
  VAR_SEQ 13: note = X is N or A
  VAR_SEQ 18: note = X is Y or W
  VAR_SEQ 20: note = X is M or Q
YYCARXLLXXXXDVDYXGXDV

SEQ ID NO: 15 - protein, 22 aa, synthetic construct
YYCARGLLRGGWNDVDYYGMDV

SEQ ID NO: 16 - protein, 22 aa, synthetic construct
YYCARGLLRGGWADVDYYGMDV

SEQ ID NO: 17 - protein, 21 aa, synthetic construct
YYCARGLLRGGADVDYYGMDV

SEQ ID NO: 18 - protein, 22 aa, synthetic construct
YYCARGLLRGAWNDVDYYGMDV

SEQ ID NO: 19 - protein, 22 aa, synthetic construct
YYCARGLLRAGWNDVDYYGMDV

SEQ ID NO: 20 - protein, 23 aa, synthetic construct
YYCARGLLLAGGWNDVDYYGMDV

SEQ ID NO: 21 - protein, 22 aa, synthetic construct
YYCARFLLRGGWNDVDYYGMDV

SEQ ID NO: 22 - protein, 22 aa, synthetic construct
YYCARGLLRGGWNDVDYYGQDV

SEQ ID NO: 23 - protein, 22 aa, synthetic construct
YYCARGLLRGGWNDVDYWGMDV

SEQ ID NO: 24 - protein, 16 aa, synthetic construct
EINHSGSTNYKTSLKS